(19) United States
(12) Patent Application Publication
(10) Pub. No.: US 2008/0249286 A1
(43) Pub. Date: Oct. 9, 2008

(54) HIGH-PRESSURE REFOLDING OF PROTEINS IN THE PRESENCE OF BINDING PARTNERS

(76) Inventors: Matthew B. SEEFELDT, Boulder, CO (US); Lyndal K. HESTERBERG, Boulder, CO (US); Theodore W. RANDOLPH, Niwot, CO (US); John F. CARPENTER, Littleton, CO (US)

Correspondence Address: MORRISON & FOERSTER LLP, 755 PAGE MILL RD, PALO ALTO, CA, 94304-1018, US

(21) Appl. No.: 12/099,009

(22) Filed: Apr. 7, 2008

Related U.S. Application Data
(60) Provisional application No. 60/922,170, filed on Apr. 5, 2007.

(51) Int. Cl. C07K 1/00; C12N 9/00

(52) U.S. Cl. 530/402; 435/183

(57) ABSTRACT

Methods for producing biologically active protein from aggregated and/or denatured proteins which comprise subjecting the protein to high hydrostatic pressure in the presence of a ligand or specific binding agent are disclosed. The ligand can be a macromolecule, such as another protein, a nucleic acid, or other macromolecules, or the ligand can be a small organic molecule.

### CROSS REFERENCE TO RELATED APPLICATIONS

This application claims priority benefit of U.S. Provisional Patent Application No. 60/922,170, filed Apr. 5, 2007. The content of that application is hereby incorporated by reference herein in its entirety.

### TECHNICAL FIELD

This invention pertains to methods for refolding proteins under high pressure in the presence of ligands or specific binding agents.

### BACKGROUND

Many proteins are valuable as therapeutic agents. Such proteins include human growth hormone, which is used to treat abnormal height when insufficient growth hormone is produced in the body, and interferon-gamma, which is used to treat neoplastic and viral diseases. Protein pharmaceuticals are often produced using recombinant DNA technology, which can enable production of higher amounts of protein than can be isolated from naturally-occurring sources, and which avoids contamination that often occurs with proteins isolated from naturally-occurring sources.

Proper folding of a protein is essential to the normal functioning of the protein. Improperly folded proteins are believed to contribute to the pathology of several diseases, including Alzheimer's disease, bovine spongiform encephalopathy (BSE, or “mad cow” disease) and human Creutzfeldt-Jakob disease (CJD), and Parkinson's disease; these diseases serve to illustrate the importance of proper protein folding.

Several proteins of therapeutic value in humans, such as recombinant human growth hormone and recombinant human interferon gamma, can be expressed in bacteria, yeast, and other microorganisms. While large amounts of proteins can be produced in such systems, the proteins are often misfolded, and often aggregate together in large clumps called inclusion bodies. The proteins cannot be used in the misfolded, aggregated state. Accordingly, methods of disaggregating and properly refolding such proteins have been the subject of much investigation. Early work in this area focused on chemical methods of disaggregating inclusion bodies (or “refractile bodies”); see, for example, U.S. Pat. No. 4,511,503.

One method of refolding proteins uses high pressure on solutions of proteins in order to disaggregate, unfold, and properly refold proteins. Such methods are described in U.S. Pat. No. 6,489,450, U.S. Pat. No. 7,064,192, U.S. Patent Application Publication No. 2004/0038333, and International Patent Application WO 02/062827. Those disclosures indicated that certain high-pressure treatments of aggregated proteins or misfolded proteins resulting in recovery of disaggregated protein retaining biological activity (i.e., the protein was properly folded, as is required for biological activity) in good yields. U.S. Pat. No. 6,489,450, U.S. Pat. No. 7,064,192, U.S. 2004/0038333, and WO 02/062827 are incorporated by reference herein in their entireties.

Certain devices have also been developed which are particularly suitable for refolding of proteins under high pressure; see U.S. Pat. App. Ser. No. 60/739,094 and International Patent Application No. PCT/US2006/045297.

While the high-pressure methods described in the patent publications above have led to significant advances in protein refolding efficiency, further improvements are being actively sought. Refolding of the human estrogen receptor-ligand binding domain at 2000 bar in the presence of 500 mM arginine and diethylstilbestrol (DES) resulted in the formation of soluble protein. (Seefeldt, Matthew B., “High pressure refolding of protein aggregates: efficacy and thermodynamics,” Ph.D. dissertation, University of Colorado at Boulder, 2004).

### DISCLOSURE OF THE INVENTION

The invention embraces methods of refolding proteins under high pressure in the presence of a ligand or specific binding agent for the protein.

In one embodiment, the invention embraces a method for producing biologically active protein from a mixture comprising aggregated and/or denatured protein, comprising adding a ligand or specific binding agent which binds to the biologically active protein to the mixture; subjecting the mixture to high hydrostatic pressure for a period of time sufficient to form biologically active protein; and reducing the pressure to atmospheric pressure; wherein the protein is biologically active at atmospheric pressure.

In another embodiment, the ligand or specific binding agent is a macromolecule. In another embodiment, the macromolecular ligand or specific binding agent is a polypeptide. In another embodiment, the macromolecular ligand or specific binding agent is a protein. In another embodiment, the macromolecular ligand or specific binding agent is a peptide. In another embodiment, the macromolecular ligand or specific binding agent is a nucleic acid, such as DNA, ssDNA, dsDNA, mRNA, tRNA, rRNA, or siRNA, or triple-stranded or multi-stranded nucleic acids. In another embodiment, the macromolecular ligand is an oligosaccharide. In another embodiment, the macromolecular ligand is a polymer. In another embodiment, the macromolecular ligand is a polymer. In another embodiment, the macromolecular ligand is a non-naturally occurring polymer.

In another embodiment, the ligand is a naturally-occurring ligand for the biologically active protein.

In one embodiment, the macromolecular ligand or specific binding agent is an immunoglobulin or antibody, or a fragment of an antibody which retains the specific binding properties of the intact antibody, such as a Fab fragment, a F(ab′)2 fragment, a Fd fragment, a Fv fragment, a dAb (single domain) fragment; isolated complementarity determining region retaining specific-antigen binding activity; and a scFv (recombinantly-produced single chain Fv) fragment. The antibody is preferably a monoclonal antibody. In another embodiment, more than one antibody can be used as a ligand during refolding, by including a combination of monoclonal antibodies. In another embodiment, more than one antibody can be used as a ligand during refolding, by using polyclonal antibodies.

In another embodiment, the macromolecular ligand or specific binding agent is a protein folding chaperone.

In another embodiment, the macromolecular ligand or specific binding agent is a polypeptide, protein, or peptide, which is not in its native conformation. In another embodiment, the non-native conformation of the polypeptide, protein, or peptide ligand is a denatured state. In another embodiment, the non-native conformation of the polypeptide, protein, or peptide ligand is an aggregated state; in one embodiment, the aggregated state is a soluble aggregate; while in another embodiment, the aggregated state is an insoluble aggregate.

In another embodiment, the specific binding agent is a small organic molecule. The small organic molecule can be a rigid small organic molecule. The small organic molecule can be a flexible small organic molecule. In another embodiment, multiple small organic molecules are added, which can be selected from rigid small organic molecules, flexible small organic molecules, or both rigid and flexible small organic molecules.

In another embodiment, the invention embraces a method for producing biologically active protein from a mixture comprising aggregated or denatured protein, comprising adding a homopolymer or non-naturally-occurring polymer which binds to the biologically active protein to the mixture; subjecting the mixture to high hydrostatic pressure for a period of time sufficient to form biologically active protein; and reducing the pressure to atmospheric pressure, wherein the protein is biologically active at atmospheric pressure.

In another embodiment, the homopolymer or non-naturally occurring polymer binds specifically to the biologically active protein. In another embodiment, the homopolymer or non-naturally occurring polymer binds preferentially to the biologically active protein over inactive, denatured, or aggregated protein. The homopolymer or non-naturally occurring polymer can bind preferentially to the biologically active protein via electrostatic interaction, or the homopolymer or non-naturally occurring polymer can bind preferentially to the biologically active protein via hydrophobic interaction. The homopolymer or non-naturally occurring polymer can be selected from heparin, dextran sulfate, polysaccharides, glycans, starch, glycogen, cellulose, or chitin.

In another embodiment, the invention embraces methods for producing biologically active protein from a mixture comprising a first aggregated or denatured protein, comprising adding a second aggregated or denatured protein to the mixture; and subjecting the mixture to high hydrostatic pressure for a period of time sufficient to form biologically active protein, wherein said first and second aggregated or denatured proteins specifically interact under high pressure; and reducing the pressure to atmospheric pressure. In another embodiment, at least one of the proteins is biologically active at atmospheric pressure. In another embodiment, both of the proteins are biologically active at atmospheric pressure. In another embodiment, the first and second proteins are then separated. The separation of the first and second proteins can be performed by affinity chromatography, HPLC, dialysis, ion exchange chromatography, size exclusion chromatography, reverse-phase chromatography, ammonium sulfate precipitation, or electrophoresis.

In one embodiment, the yield of biologically active protein for at least one of the first and second aggregated or denatured proteins, after subjecting the mixture to high hydrostatic pressure for a period of time sufficient to form biologically active protein, is higher when the proteins are together in the mixture than if the first and second proteins were in separate mixtures. In another embodiment, the yield is at least about 10% higher. In another embodiment, the yield is at least about 25% higher. In another embodiment, the yield is at least about 50% higher. In another embodiment, the yield is at least 100% higher. In another embodiment, the yield is at least 200% higher.

In one embodiment, one of the first or second proteins is a chaperone protein. In another embodiment, the first and second proteins continue to interact after the pressure is reduced to atmospheric pressure. In another embodiment, the first and second proteins form a heterodimer in their biologically active state. In another embodiment, one of the first and second proteins is an enzyme and the other is a substrate for the enzyme. In another embodiment, one of the first and second proteins is an enzyme and the other is an inhibitor of the enzyme. In another embodiment, one of the first and second proteins is an enzyme and the other is a regulator or modulator of the enzyme. In another embodiment, one of the first and second proteins is a receptor and the other is a ligand for the receptor. In another embodiment, one of the first and second proteins is a receptor and the other is an agonist for the receptor. In another embodiment, one of the first and second proteins is a receptor and the other is an antagonist for the receptor. In another embodiment, one of the first and second proteins is a monoclonal antibody and the other is a protein containing an epitope to which the monoclonal antibody binds. In another embodiment, one of the proteins is a growth factor and the other is a binding protein for that growth factor. In another embodiment, one of the proteins is a kinase and the other is the protein phosphorylated by the kinase.

In certain embodiments, the high hydrostatic pressure is from about 500 to about 10,000 bar. In some variations, the increased hydrostatic pressure is from about 1500 to about 4000 bar. In particular variations, the increased hydrostatic pressure is about 2000 bar.

In some embodiments, reducing the pressure to atmospheric pressure comprises stepwise pressure reductions. In certain variations, during at least one pressure reduction step the rate of pressure reduction is from about 5000 bar/4 days to about 5000 bar/sec. In some embodiments, during at least one pressure reduction step the rate of pressure reduction is about 250 bar/5 minutes. In some embodiments there are at least 2 stepwise pressure reductions. In additional embodiments, there are more than 2 stepwise pressure reductions.

In some embodiments, the step of reducing the pressure to atmospheric pressure further comprises a hold period at constant pressure after at least one of the stepwise pressure reductions. In some embodiments, the hold period is from about 2 hours to about 50 hours. In certain embodiments, the hold period is about 6 hours. In some embodiments, during the hold period the constant pressure is from about 500 bar to about 2000 bar. In certain embodiments, during the hold period the constant pressure is about 1000 bar. In certain embodiments, during the hold period the constant pressure is about one-half of the initial high hydrostatic pressure. In some embodiments, during the hold period the constant pressure is about one-third of the initial high hydrostatic pressure.

In some embodiments, the step of reducing the pressure includes a continuous rate of pressure reduction. In some embodiments, the rate of pressure reduction is from about 5000 bar/1 sec to about 5000 bar/4 days. In certain embodiments, the rate of pressure reduction is 250 bar/5 minutes.

In certain embodiments the methods further include, at any point in the method, adding one or more disulfide shuffling agent pairs to the mixture in an amount sufficient to facilitate formation of native disulfide bonds in the protein. In one embodiment, the method comprises subjecting the mixture to a further period of increased hydrostatic pressure compared to atmospheric pressure for a time sufficient for formation of native disulfide bonds in the protein.

In some embodiments, the pH of the mixture is from about pH 4 to about pH 12. In certain embodiments, the pH of the mixture is from about pH 6 to about pH 8.5. In particular embodiments, the pH of the mixture is from about pH 7 to about pH 8.5.

In some embodiments, the step of subjecting the mixture to high pressure is performed at a temperature from about 0° C. to about 100° C. In some variations, the step of subjecting the mixture to high pressure is performed at a temperature from about 0° C. to about 75° C.

In some embodiments, the mixture further comprises one or more additional agents selected from one or more stabilizing agents, one or more buffering agents, one or more surfactants, one or more disulfide shuffling agent pairs, one or more chaotropic agents, one or more salts, and combinations of two or more of the foregoing.

In certain variations, the one or more additional agents is one or more stabilizing agents. In some embodiments, the one or more stabilizing agents is selected from one or more free amino acids, one or more preferentially excluding compounds, trimethylamine oxide, one or more cyclodextrans, one or more molecular chaperones, and combinations of two or more of the foregoing.

In some embodiments, the one or more stabilizing agents is one or more preferentially excluding compounds. In certain embodiments, the one or more preferentially excluding compounds is one or more sugars, glycerol, hexylene glycol, or combinations of two or more of the foregoing. In one embodiment, the one or more stabilizing agents is hexylene glycol. In some embodiments, the one or more preferentially excluding compounds is one or more sugars. In certain embodiments, the one or more sugars is sucrose, trehalose, dextrose, mannose, or combinations of two or more of the foregoing. In some variations, the one or more sugars is present at a concentration of from about 0.1 mM to about the solubility limit of the sugar.

In some embodiments, the one or more stabilizing agents is one or more free amino acids. In certain variations, the one or more free amino acids is arginine, lysine, proline, glutamine, glycine, histidine, or combinations of two or more of the foregoing. In some embodiments, the one or more free amino acids is present at a concentration of from about 0.1 mM to about the solubility limit of the free amino acid.

In some variations, the one or more stabilizing agents is a cyclodextran. In some embodiments, the cyclodextran is present at a concentration of from about 0.1 mM to about the solubility limit of the cyclodextran.

In some variations, the one or more stabilizing agents is a molecular chaperone. In certain variations the molecular chaperone is GroEs or GroEL. In some variations, the molecular chaperone is present at a concentration of from about 0.01 mg/ml to about 10 mg/ml.

In some embodiments, the one or more additional agents is one or more surfactants. In particular embodiments, the one or more surfactants is selected from polysorbates, polyoxyethylene ethers, alkyltrimethylammonium bromides, alkyltrimethyl ammonium chlorides, pyranosides and combinations of two or more of the foregoing. In some variations, the one or more surfactants is selected from polysorbate 80, polysorbate 20, Triton X-100, Brij 35, sarcosyl, octyl phenol ethoxylate, β-octyl-gluco-pyranoside, polyoxyethyleneglycol dodecyl ether, sodium dodecyl sulfate, polyethoxysorbitan, deoxycholate, sodium octyl sulfate, sodium tetradecyl sulfate, sodium cholate, octylthioglucopyranoside, n-octylglucopyranoside, octylphenoxypolyethoxy-ethanol, polyoxyethylene sorbitan, cetylpyridinium chloride, and sodium bis (2-ethylhexyl) sulfosuccinate.

In some variations, the one or more additional agents is one or more buffering agents. In certain variations, the one or more buffering agents is an organic buffer. In particular variations, the one or more buffering agents is an inorganic buffer. In some embodiments, the one or more buffering agents is selected from phosphate buffers, carbonate buffers, citrate, Tris, MOPS, MES, acetate, and HEPES.

In some embodiments, the one or more additional agent is one or more chaotropic agents. In some variations, the one or more chaotropic agents is guanidine, guanidine sulfate, guanidine hydrochloride, urea, or thiocyanate. In some embodiments, the one or more chaotropic agents is urea in a concentration from about 0.1 mM to about 8 M. In some embodiments, the concentration of urea is about 0.1 M to about 2.0 M, or about 0.5 M, about 1.0 M, about 1.5 M, or about 2.0 M. In certain embodiments, the one or more chaotropic agents is guanidine hydrochloride in a concentration of from about 0.1 mM to about 8 M. In some embodiments, the concentration of guanidine hydrochloride is about 0.1 M to about 2.0 M, or about 0.5 M, about 1.0 M, about 1.5 M, or about 2.0 M.

In some variations, the one or more additional agents are one or more disulfide shuffling agent pair. In some embodiments, the disulfide shuffling agent pair include an oxidizing agent and a reducing agent. In some variations, the oxidizing agent is at least one of oxidized glutathione, cystine, cystamine, molecular oxygen, iodosobenzoic acid, sulfitolysis or a peroxide and the reducing agent is at least one of glutathione, cysteine, cysteamine, diothiothreitol, dithioerythritol, tris(2-carboxyethyl)phosphine hydrochloride, or β-mercaptoethanol. In certain variations, the disulfide shuffling agent pair is present at an oxidized concentration of from about 0.1 mM to about 100 mM oxidized thiol. In some variations, the concentration is from about 0.1 mM to about 10 mM. In certain variations, the concentration is from about 2 mM to about 6 mM.

In some embodiments, the time during which the mixture is subjected to high hydrostatic pressure is from about 15 minutes to about one week. In some embodiments, the time is from about 15 minutes to about 50 hours. In some embodiments, the time is from about 6 hours to about 18 hours.

In some embodiments, the protein is a monomer. In certain embodiments, the protein is a dimer. In particular embodiments, the dimer is a homodimer. In some variations, the dimer is a heterodimer. In some embodiments, the protein is a trimer. In particular embodiments, the trimer is a homotrimer. In certain embodiments, the trimer is a heterotrimer. In particular embodiments, the protein is a tetramer. In particular embodiments, the tetramer is a homotretramer. In some variations, the tetramer is a heterotetramer.

In some variations, the total concentration of protein in the mixture is from about 0.01 mg/mL to about 300 mg/mL. In some embodiments, the total concentration of protein in the mixture is from about 0.01 mg/mL to about 150 mg/mL.

In some embodiments, the method further includes agitation of the mixture during the period of high hydrostatic pressure.

In certain embodiments where chaotropic agents are included, the methods further include the step of reducing the amount of the chaotropic agent present in the mixture after the high hydrostatic pressure.

In some variations, the method does not include any chaotropic agent.

In one variation of any of the foregoing methods, the invention embraces a method for producing biologically active protein from a mixture comprising aggregated and/or denatured protein, comprising subjecting the mixture to high hydrostatic pressure; adding a ligand or specific binding agent which binds to the biologically active protein to the mixture while the mixture is subjected to high hydrostatic pressure; optionally waiting for a period of time sufficient to form biologically active protein; and reducing the pressure to atmospheric pressure; wherein the protein is biologically active at atmospheric pressure.

The invention also embraces proteins prepared by any of the methods described herein.

### DETAILED DESCRIPTION OF THE INVENTION

By “small organic molecule” is meant an organic molecule having a molecular weight of about 600 or lower.

By “polymer” is meant a molecule made up of multiple individual units, of molecular weight generally 1000 or higher. Among other differences, polymers often have multiple conformations in solution which have small differences in free energy between conformations, whereas small organic molecules often have very few conformations in solution, which may differ greatly in free energy.

As used herein, a “protein aggregate” is defined as being composed of a multiplicity of protein molecules wherein non-native noncovalent interactions and/or non-native covalent bonds (such as non-native intermolecular disulfide bonds) hold the protein molecules together. Typically, but not always, an aggregate contains sufficient molecules so that it is insoluble; such aggregates are insoluble aggregates. There are also oligomeric proteins which occur in aggregates in solution; such aggregates are soluble aggregates. In addition, there is typically (but not always) a display of at least one epitope or region on the aggregate surface which is not displayed on the surface of native, non-aggregated protein. “Inclusion bodies” are a type of aggregate of particular interest to which the present invention is applicable. Other protein aggregates include, but are not limited to, soluble and insoluble precipitates, soluble non-native oligomers, gels, fibrils, films, filaments, protofibrils, amyloid deposits, plaques, and dispersed non-native intracellular oligomers.

“Atmospheric,” “ambient,” or “standard” pressure is defined as approximately 15 pounds per square inch (psi) or approximately 1 bar or approximately 100,000 Pascals.

“Biological activity” of a protein or polypeptide as used herein, means that the protein or polypeptide retains at least about 10% of maximal known specific activity as measured in an assay that is generally accepted in the art to be correlated with the known or intended utility of the protein. For proteins or polypeptides intended for therapeutic use, the assay of choice is one accepted by a regulatory agency to which data on safety and efficacy of the protein or polypeptide must be submitted. In some embodiments, a protein or polypeptide having at least about 10% of maximal known specific activity or of the non-denatured molecule is “biologically active” for the purposes of the invention. In some embodiments, the biological activity is at least about 15%, at least about 20%, at least about 25%, at least about 30%, at least about 40%, at least about 50%, at least about 75%, or at least about 90% of maximal known specific activity or of the non-denatured molecule.

“Denatured,” as applied to a protein in the present context, means that native secondary, tertiary, and/or quartenary structure is disrupted to an extent that the protein does not have biological activity.

In contrast to “denatured,” the “native conformation” of a protein refers to the secondary, tertiary and/or quaternary structures of a protein in its biologically active state.

“Refolding” in the present context means the process by which a fully or partially denatured polypeptide adopts secondary, tertiary and quaternary structure like that of the cognate native molecule. A properly refolded polypeptide has biological activity that is at least about 10% of the non-denatured molecule, preferably biological activity that is substantially that of the non-denatured molecule. In some embodiments, the biological activity is at least about 15%, about 20%, about 25%, about 30%, about 40%, about 50%, about 75%, or about 90% of the non-denatured molecule. Where the native polypeptide has disulfide bonds, oxidation to form native disulfide bonds is a desired component of the refolding process.

Thermodynamic Analysis of Refolding with Specific Binding Agents

Cosolvent binding improves protein pressure refolding through two probable mechanisms. The effect of a cosolvent on any equilibrium at a given concentration of cosolvent, is expressed by the Wyman linkage equation:

$$\frac{\partial ln K}{\partial ln {a}_{3}} = {( \frac{\partial {m}_{3}}{\partial {m}_{2}} )}_{T , P , μ}^{D} - {( \frac{\partial {m}_{3}}{\partial {m}_{2}} )}_{T , P , μ}^{N}$$

where a₃ is the activity of component 3, the cosolvent, m₂ and m₃ refer to the concentration of protein and cosolvent respectively, and D and N refer specifically to the denaturation equilibrium N [symbol] D (Xie, G. F. and S. N. Timasheff, Biophysical Chemistry 64(1-3): 25-43 (1997)). Since the cosolvent binds only to the native conformation and presumably cannot bind to the denatured state, Xie and Timasheff conclude that the formation of the native state is favored as the activity of the cosolvent increases in solution (see Xie and Timasheff, 1997). Note that Xie and Timasheff used trehalose in their study, a molecule which does not bind specifically to the protein studied; hence their reference to trehalose as a cosolvent.

One embodiment of the present invention provides for enhanced protein refolding in the presence of a specific binding agent or cofactor, in contrast to the general “cosolvent” used in Xie and Timasheff's work. Instead of the denatured state analyzed in Xie and Timasheff, this embodiment of the invention postulates an equilibrium between native and aggregate proteins, where the presence of a specific binding cofactor can favor refolding under high hydrostatic pressures. The presence of a specific binding cofactor in the refolding solution increases the stability of the native conformation, thermodynamically adjusting the equilibrium to favor the native conformation and concomitantly improving refolding yields.

A second, more subtle mechanism can also contribute to enhanced yields of native protein while using cofactor binding under high pressure. Cofactor binding has been shown to decrease the adiabatic compressibility of native proteins (Kamiyama, T. and K. Gekko, Biochimica Et Biophysica Acta-Protein Structure and Molecular Enzymology 1478(2): 257-266 (2000); Gekko, K., Biochimica Et Biophysica Acta-Protein Structure and Molecular Enzymology 1595(1-2): 382-386 (2002); Taulier, N. and T. V. Chalikian, Biochimica Et Biophysica Acta-Protein Structure and Molecular Enzymology 1595(1-2): 48-70 (2002). This parameter is derived from volume contributions arising from hydration effects and the presence of solvent-free cavities due to imperfect packing within the protein interior (Gekko, K. and Y. Hasegawa, Biochemistry 25(21): 6563-6571 (1986)). Decreases in adiabatic compressibility are also correlated to decreases in protein specific volume (Gekko, K. and Y. Hasegawa, Biochemistry 25(21): 6563-6571 (1986)), and consequently cofactor binding should promote a more dense native conformation. Since pressure-modulated refolding is driven thermodynamically by the decreases in volume upon native-state formation, the ΔV_{refolding} is decreased and promotes refolding under high hydrostatic pressure.

### Proteins for Refolding

Proteins which can be refolded with the methods of the invention include a wide variety of polypeptide or polypeptide-containing molecules. Proteins which can be refolded include both globular and fibrous proteins; the preferred protein is a globular protein. Proteins which can be refolded include, but are not limited to, monomeric, dimeric, multimeric, heterodimeric, heterotrimeric, and heterotetrameric proteins; disulfide bonded proteins; glycosylated proteins; helical proteins; and alpha helix- and beta sheet-containing proteins. Examples of particular proteins include, but are not limited to, hormones, antibodies, enzymes, and metal binding proteins. Examples of protein structures which can be refolded include up-and-down helix bundle, Greek key helix bundle, miscellaneous antiparallel alpha helix, singly wound parallel beta barrel, doubly wound parallel beta sheet, miscellaneous parallel alpha/beta, up-and-down beta barrel (antiparallel beta), Greek key beta barrel (antiparallel beta), multiple, partial, and other beta barrel, open-face beta sandwich, miscellaneous antiparallel beta, SS-rich (disulfide-rich), and metal-rich proteins.

“Heterologous” proteins are proteins which are normally not produced by a particular host cell. Recombinant DNA technology has permitted the expression of relatively large amounts of heterologous proteins (for example, growth hormone) from transformed host cells such as E. coli. These proteins are often sequestered in insoluble inclusion bodies in the cytoplasm and/or periplasm of the host cell. The inclusion bodies or cytoplasmic aggregates contain, at least in part, the heterologous protein to be recovered. These aggregates often appear as bright spots under a phase contrast microscope. A host cell is a microbial cell, such as bacteria or fungi (e.g., yeast) or other suitable cells including an animal cell or a plant cell, that has been transformed to express the heterologous polypeptide of interest.

### Co-Refolding of Denatured Interacting Proteins

In one embodiment of the invention, two or more proteins are simultaneously refolded from the denatured or aggregated state. The proteins may not interact with each other, either in the denatured or aggregated state, or in the refolded, biologically active state. In this case, the proteins may be folded together due to the convenience of running one refolding experiment rather than separate experiments, or the proteins may be refolded together because they are expressed or produced together in the denatured or aggregated state. The proteins may be separated from each other using methods known in the art. Alternatively, some uses may permit or even require the presence of multiple non-interacting proteins, such as combinatorial screening of enzymes or binding agents.

In another embodiment of the invention, two or more proteins are simultaneously refolded from the denatured or aggregated state, and the proteins do interact with each other, either in the denatured or aggregated state, or in the refolded, biologically active state. In this embodiment, the two or more denatured proteins may be two or more components of a heteromultimeric protein (e.g., a heterodimer, a heterotrimer, etc.); the two or more denatured proteins may be an enzyme and a protein substrate for the enzyme; the two or more denatured proteins may be an enzyme and a protein inhibitor for the enzyme; the two or more denatured proteins may be an enzyme and a protein that regulates or modulates the enzyme by a specific binding interaction with the enzyme; the two or more denatured proteins may be a receptor and a protein ligand for the receptor; the two or more denatured proteins may be a receptor and a protein agonist for the receptor; or the two or more denatured proteins may be a receptor and a protein antagonist for the receptor. The two or more denatured or aggregated proteins may interact while in the denatured or aggregated state (e.g., before high pressure is applied); they may interact during the pressurization of the mixture; they may interact during the incubation under pressure; they may interact during the depressurization of the mixture; they may interact at atmospheric pressure, or they may interact during any combination of, or all of, those stages. The refolding of a denatured or aggregated protein is carried out in the presence of another one or more denatured or aggregated proteins in order to enhance the yield of biologically active proteins of any one of, any combination of, or all of the denatured or aggregated proteins. In various embodiments, the yield of biologically active protein of any one or more of the denatured or aggregated proteins is enhanced by 5%, 10%, 15%, 20%, 25%, 30%, 40%, 50%, 60%, 70%, 80%, 90%, or 100% over the yield of biologically active protein that would have resulted if the proteins were folded separately.

For any given protein of interest, a suitable proteinaceous binding partner will often already be known; alternatively, the scientific literature and various databases can be searched for suitable binding partners. Several databases track protein interactions and can be consulted to identify binding partners for proteins of interest for refolding. Some of these databases are listed as follows: the MIPS Mammalian Protein-Protein Interaction Database, located at URL mips.gsf.de/proj/ppi (Pagel, P. et al., Bioinformatics 21(6):832-834 (2005)) collects protein-protein interaction data from the scientific literature, and additionally provides links to several other protein-protein interaction databases; the Human Protein Reference Database (HPRD), located at URL World-Wide-Web.hprd.org, provides information regarding protein interaction networks (and much additional information) for each protein in the human proteome (see Peri, S. et al., Genome Research, 13:2363-2371 (2003)); and BioGRID, located at URL World-Wide-Web.thebiogrid.org, is a database of protein and genetic interactions. While an exhaustive list of interacting proteins and protein-protein binding pairs would be impractical to include herein, the following table lists various binding pairs of particular interest which can be refolded using the methods of the invention. The skilled artisan will understand that, while the table is in the format of “protein” and “binding partner,” each row can be reversed; e.g., while VEGF is listed as the protein (to be refolded) and VEGF receptor 1 is listed as a binding partner, the opposite relation can also hold, where VEGF receptor 1 is the protein to be refolded and VEGF is its binding partner. In certain cases, both the protein and the binding partner are of interest to be refolded.

| Protein | Binding Partner |
|---|---|
| Vascular endothelial growth | VEGF receptor 1; VEGF receptor 2; |
| factor (VEGF) | neuropilins |
| Insulin-like growth factor (IGF) | IGF binding protein (e.g., IGFBP-3) |
| (e.g., IGF-1) | |
| Insulin-like binding protein-5 | RAS associated domain binding |
| (IGFBP-5) | protein (RASSFIC) |
| Profilin | Paladin |
| Fibroblast growth factor 7 | Fibroblast binding protein |
| (FGF-7), FGF-10, FGF-22 | |
| myosin 1Xb | BIG1 (brefeldin A-inhibited guanine |
| | nucleotide exchange factor 1) |
| TAK1 (transforming growth | TAB3 (TAK1-binding protein-3) |
| factor-beta-activated protein | |
| kinase) | |

### Ligands for Use in Refolding

Various ligands can be used in refolding of proteins. Such ligands include, but are not limited to, antibodies, receptors, peptides, peptidomimetics, vitamins, cofactors, prosthetic groups, substrates, products, competitive inhibitors, metals and other small or large molecules.

One particular group of ligands useful in the invention is the group of small organic molecules. Small organic molecules in turn may be divided into two types: rigid small organic molecules and flexible small organic molecules. A rigid molecule typically has one predominant conformation in solution; a rigid molecule is defined as having greater than about 50%, greater than about 75%, or greater than about 90% of the molecules in solution present in a single conformation. A flexible molecule has multiple solution conformations, where a single conformation accounts for no more than about 50%, no more than about 25%, or no more than about 10% of the molecules in solution. These conformational populations are preferably measured in aqueous solution, more preferably in the refolding buffer to be used for the protein(s) of interest. A variety of methods can be used to define different conformations of a molecule for the purposes of the foregoing conditions. Experimental methods include, but are not limited to, detection of different conformations by nuclear magnetic resonance spectra (conformations that interconvert slowly on the NMR time scale can be detected by NMR, although rapidly exchanging conformations typically cannot be detected by ordinary NMR experiments), fluorescence spectroscopy, or other spectroscopic methods. Computational methods include, but are not limited to, molecular mechanics and molecular dynamics simulations where molecules display different conformations (e.g., conformations with root-mean-square deviations differing by 1, 2, 3, or 4 Angstroms, or conformations with differences of 0.25, 0.5, 0.75, 1, 2, 3, 4, or 5 kcal in free energy).

Antibodies raised against a specific protein target of interest can also be used during high-pressure refolding of proteins of interest. Antibodies can be of either polyclonal or monoclonal origin. In some instances, polyclonal antibodies will be useful, e.g., for providing antibodies which bind to distinct epitopes on the same protein and/or distinct antibodies binding to the same epitope on the protein, in order to provide multiple folding “guidance,” while in other instances, monoclonal antibodies will be useful, e.g. for preparation of well-characterized folding conditions. Either intact antibodies or antibody fragments which retain the antigen-binding properties of the intact antibody can be used. Such antibody fragments include Fab fragments, F(ab′)2 fragments, Fd fragments, Fv fragments, dAb (single domain) fragments (Ward et al., Nature 341:544 (1989)); isolated complementarity determining regions retaining specific-antigen binding activity; and scFv (recombinantly-produced single chain Fv) fragments.

### Pressure-Assisted Refolding in the Presence of Homopolymers and Non-Naturally-Occurring Polymers

Co-refolding can also be carried out in the presence of homopolymers and non-naturally occurring polymers. Examples of homopolymers include heparin, dextran, dextran sulfate, polysaccharides or glycans such as starch, glycogen, cellulose, chitin, and other polymers. Other homopolymers include polyglutamate (polyanionic), polyaspartate (polyanionic), polylysine (polycationic), and other homopolymers of amino acids.

Proteins which bind to polyanions can be refolded in the presence of polyanions (e.g., heparin or dextran sulfate). Concentrations of polyanion can range from about 1 ug/mL to about 10 mg/mL, or about 10 ug/mL to about 1 mg/mL, or about 10 ug/mL to about 100 ug/mL. Alternatively, the molar ratio of the protein to be refolded to the polyanion can be about 100:1, about 50:1, about 30:1, about 20:1, about 10:1, about 5:1, about 4:1, about 3:1, about 2:1, about 1:1, about 1:2, about 1:3, about 1:4, about 1:5, about 1:10, about 1:20, about 1:30, about 1:50, or about 1:100. Ranges from about 100:1 to about 50:1, about 75:1 to about 25:1, about 50:1 to about 10:1, about 10:1 to about 1:1, about 10:1 to about 1:10, about 1:1 to about 1:10, about 10:1 to about 50:1, about 25:1 to about 75:1, or about 50:1 to about 100:1 can also be employed. Considerations in the concentration of polyanion to employ include the binding constant of the polyanion to the protein of interest. The polyanion should be readily removable from the binding surface of the protein after removal of the polyanion from the bulk solution.

An example of a protein that binds effectively to polyanions is Fibroblast Growth Factor-10 (FGF-10) (Kamerzell et al., Biochemistry 45:15288-15300 (2006)). FGF-10 is a member of the FGF family of structurally related heparin/polyanion binding proteins involved in a variety of physiological and pathological processes including morphogenesis, angiogenesis, and carcinogenesis. FGF-10 activity and specificity is modulated by heparin binding, heparin sulfate binding, and oversulfated chondroitin sulfate-E, all polyanions. Kamerzell et al. (Biochemistry 45:15288-15300 (2006)) show that the stability of FGF-10 is increased by the addition of phytic acid, heparin, and dextran sulfate. Consequently, high pressure refolding of FGF-10 in the presence of a polyanion should improve refolding yields. Other proteins which may be refolded in the presence of polyanions include, but are not limited to, vascular endothelial growth factor (VEGF) and fibroblast growth factor-1 (FGF-1). Polyanions that can be included during the high pressure refolding include, but are not limited to, heparin, dextran sulfate, dermatan sulfate, pentosan polysulfate, sulfated bis-lactobionic acid amide, sulfated bis-maltobionic acid amide, fucosylated chondroitin sulfate, chondroitin sulfate, and fucoidan. Kajio et al. demonstrated that dextran sulfate protected basic fibroblast growth factor (bFGF) from heat and acid inactivation and from proteolytic degradation (see FEBS Lett. 306:243 (1992)).

### Protein Purification

A wide variety of techniques are known in the art for protein separation and purification, such as affinity chromatography, high-pressure liquid chromatography (HPLC), dialysis, ion exchange chromatography, size exclusion chromatography, reverse-phase chromatography, ammonium sulfate precipitation, or electrophoresis. Several conditions for HPLC can be varied for enhancing separation, such as the stationary and mobile phases. HPLC can be used with ion-exchange columns, reverse-phase columns, affinity columns, size-exclusion columns, and other types of columns. FPLC, or “Fast Performance Liquid Chromatography,” can also be used. Gel-filtration chromatography can be used at low solvent pressures. Removal of small molecules (such as chaotropes, kosmotropes, surfactants, detergents, reducing agents, oxidizing agents, or small molecule binding partners) from protein solutions can be achieved via diafiltration, ultrafiltration, or dialysis.

For refolding in the presence of ligands, the protein can be present in varying degrees of purity, for example, as part of a whole cell slurry, a partially purified cell preparation, a partially purified preparation of inclusion bodies, or a precipitated partially purified protein. Further purification of the protein can be performed after refolding if necessary.

### Other Considerations

Several conditions can be adjusted for optimal protein refolding.

Protein Concentration: the concentration of protein can be adjusted for optimal protein refolding. One advantage of high-pressure protein refolding is that much higher concentrations of protein can be used as compared to chemical refolding techniques. Protein concentrations of at least about 0.1 mg/ml, at least about 1.0 mg/ml, at least about 5.0 mg/ml, at least about 10 mg/ml, or at least about 20 mg/ml can be used. Protein in the mixture may be present in a concentration of from about 0.001 mg/ml to about 300 mg/ml. Thus, in some embodiments the protein is present in a concentration of from about 0.001 mg/ml to about 250 mg/ml, from about 0.001 mg/ml to about 200 mg/ml, from about 0.001 mg/ml to about 150 mg/ml, from about 0.001 mg/ml to about 100 mg/ml, from about 0.001 mg/ml to about 50 mg/ml, from about 0.001 mg/ml to about 30 mg/ml, from about 0.05 mg/ml to about 300 mg/ml, from about 0.05 mg/ml to about 250 mg/ml, from about 0.05 mg/ml to about 200 mg/ml, from about 0.05 mg/ml to about 150 mg/ml, from about 0.05 mg/ml to about 100 mg/ml, from about 0.05 mg/ml to about 50 mg/ml, from about 0.05 mg/ml to about 30 mg/ml, from about 10 mg/ml to about 300 mg/ml, from about 10 mg/ml to about 250 mg/ml, from about 10 mg/ml to about 200 mg/ml, from about 10 mg/ml to about 150 mg/ml, from about 10 mg/ml to about 100 mg/ml, from about 10 mg/ml to about 50 mg/ml, from about 10 mg/ml to about 30 mg/ml, from about 0.1 mg/ml to about 100 mg/ml, from about 0.1 mg/ml to about 10 mg/ml, from about 1 mg/ml to about 100 mg/ml, from about 1 mg/ml to about 10 mg/ml, from about 10 mg/ml to about 100 mg/ml, or from about 50 mg/ml to about 100 mg/ml can be used.

As used in the present context the phrase “a period of time sufficient to form biologically active protein” and cognates thereof refer to the time needed for the protein aggregates to be disaggregated and to adopt a conformation where the protein is biologically active. Typically, the time sufficient for solubilization is about 15 minutes to about 50 hours, or possibly longer depending on the particular protein, (e.g., as long as necessary for the protein; for example, up to about 1 week, about 5 days, about 4 days, about 3 days, etc.). Thus, in some embodiments of the methods, the time sufficient for formation of biologically active protein may be from about 2 to about 30 hours, from about 2 to about 24 hours, from about 2 to about 18 hours, from about 1 to about 10 hours, from about 1 to about 8 hours, from about 1 to about 6 hours, from about 2 to about 10 hours, from about 2 to about 8 hours, from about 2 to about 6 hours, or about 2 hours, about 6 hours, about 10 hours, about 16 hours, about 20 hours, or about 30 hours, from about 2 to about 10 hours, from about 2 to about 8 hours, from about 2 to about 6 hours, from about 12 to about 18 hours, or from about 10 to about 20 hours.

The mixture comprising protein aggregates or denatured protein is typically an aqueous solution or aqueous suspension. The mixture may also include other components. These additional components may be one or more additional agents including: one or more stabilizing agents, one or more buffering agents, one or more surfactants, one or more disulfide shuffling agent pairs, one or more salts, one or more chaotropes, or combinations of two or more of the foregoing.

The amounts of the additional agents will vary depending on the selection of the protein, however, the effect of the presence (and amount) or absence of each additional agent or combinations of agents can be determined and optimized using the teachings provided herein.

Exemplary additional agents include, but are not limited to, buffers (examples include, but are not limited to, phosphate buffer, borate buffer, carbonate buffer, citrate buffer, HEPES, MEPS), salts (examples include, but are not limited to, the chloride, sulfate, and carbonate salts of sodium, zinc, calcium, ammonium and potassium), chaotropes (examples include, but are not limited to, urea, guanidine hydrochloride, guanidine sulfate and sarcosine), and stabilizing agents (e.g., preferential excluding compounds, etc.).

Non-specific protein stabilizing agents act to favor the most compact conformation of a protein. Such agents include, but are not limited to, one or more free amino acids, one or more preferentially excluding compounds, kosmotropes, trimethylamine oxide, cyclodextrans, molecular chaperones, and combinations of two or more of the foregoing.

Amino acids can be used to prevent reaggregation and facilitate the dissociation of hydrogen bonds. Typical amino acids that can be used, but not limited to, are arginine, lysine, proline, glycine, histidine, and glutamine or combinations of two or more of the foregoing. In some embodiments, the free amino acid(s) is present in a concentration of about 0.1 mM to about the solubility limited of the amino acid, and in some variations from about 0.1 mM to about 2 M. The optimal concentration is a function of the desired protein and should favor the native conformation.

Preferentially excluding compounds can be used to stabilize the native confirmation of the protein of interest. Possible preferentially excluding compounds include, but are not limited to, sucrose, hexylene glycol, sugars (e.g., sucrose, trehalose, dextrose, mannose), and glycerol. The range of concentrations that can be use are from 0.1 mM to the maximum concentration at the solubility limit of the specific compound. The optimum preferential excluding concentration is a function of the protein of interest.

In particular embodiments, the preferentially excluding compound is one or more sugars (e.g., sucrose, trehalose, dextrose, mannose or combinations of two or more of the foregoing). In some embodiments, the sugar(s) is present in a concentration of about 0.1 mM to about the solubility limit of the particular compound. In some embodiments, the concentration is from about 0.1 mM to about 2M, from about 0.1 mM to about 1.5M, from about 0.1 mM to about 1M, from about 0.1 mM to about 0.5M, from about 0.1 mM to about 0.3M, from about 0.1 mM to about 0.2 M, from about 0.1 mM to about 0.1 mM, from about 0.1 mM to about 50 mM, from about 0.1 mM to about 25 mM, or from about 0.1 mM to about 10 mM.

In some embodiments, the stabilizing agent is one or more of sucrose, trehalose, glycerol, betaine, amino acid(s), or trimethylamine oxide.

In certain embodiments, the stabilizing agent is a cyclodextran. In some embodiments, the cyclodextran is present in a concentration of about 0.1 mM to about the solubility limit of the cyclodextran. In some variations from about 0.1 mM to about 2 M.

In certain embodiments, the stabilizing agent is a molecular chaperone. In some embodiments, the molecular chaperone is present in a concentration of about 0.01 mg/ml to 10 mg/ml.

A single stabilizing agent maybe be used or a combination of two or more stabilizing agents (e.g., at least two, at least three, or 2 or 3 or 4 stabilizing agents). Where more than one stabilizing agent is used, the stabilizing agents may be of different types, for example, at least one preferentially excluding compound and at least one free amino acid, at least one preferentially excluding compound and betaine, etc.

Buffering agents may be present to maintain a desired pH value or pH range. Numerous suitable buffering agents are known to the skilled artisan and should be selected based on the pH that favors (or at least does not disfavor) the native conformation of the protein of interest. Either inorganic or organic buffering agents may be used. Suitable concentrations are known to the skilled artisan and should be optimized for the methods as described herein according to the teaching provided based on the characteristics of the desired protein.

Thus, in some embodiments, at least one inorganic buffering agent is used (e.g., phosphate, carbonate, etc.). In certain embodiments, at least one organic buffering agent is used (e.g., citrate, acetate, Tris, MOPS, MES, HEPES, etc.) Additional organic and inorganic buffering agents are well known to the art.

In some embodiments, the one or more buffering agents is phosphate buffer, borate buffer, carbonate buffer, citrate buffer, HEPES, MEPS, MOPS, MES, or acetate buffer.

In some embodiments, the one or more buffering agents is phosphate buffers, carbonate buffers, citrate, Tris, MOPS, MES, acetate or HEPES.

A single buffering agent maybe be used or a combination of two or more buffering agents (e.g., at least two, at least 3, or 2 or 3 or 4 buffering agents).

A “surfactant” as used in the present context is a surface active compound which reduces the surface tension of water.

Surfactants are used to improve the solubility of certain proteins. Surfactants should generally be used at concentrations above or below their critical micelle concentration (CMC), for example, from about 5% to about 20% above or below the CMC. However, these values will vary dependent upon the surfactant chosen, for example, surfactants such as, beta-octylgluco-pyranoside may be effective at lower concentrations than, for example, surfactants such as TWEEN-20 (polysorbate 20). The optimal concentration is a function of each surfactant, which has its own CMC.

Useful surfactants include nonionic (including, but not limited to, t-octylphenoxypolyethoxy-ethanol and polyoxyethylene sorbitan), anionic (e.g., sodium dodecyl sulfate) and cationic (e.g., cetylpyridinium chloride) and amphoteric agents. Suitable surfactants include, but are not limited to deoxycholate, sodium octyl sulfate, sodium tetradecyl sulfate, polyoxyethylene ethers, sodium cholate, octylthioglucopyranoside, n-octylglucopyranoside, alkyltrimethylammonium bromides, alkyltrimethyl ammonium chlorides, and sodium bis (2 ethylhexyl) sulfosuccinate. In some embodiments the surfactant may be polysorbate 80, polysorbate 20, sarcosyl, Triton X-100, β-octyl-gluco-pyranoside, or Brij 35.

In some embodiments the one or more surfactant may be a polysorbate, polyoxyethylene ether, alkyltrimethylammonium bromide, pyranosides or combination of two or more of the foregoing. In certain embodiments, the one or more surfactant may be β-octyl-gluco-pyranoside, Brij 35, or a polysorbate.

In certain embodiments the one or more surfactant may be octyl phenol ethoxylate, β-octyl-gluco-pyranoside, polyoxyethyleneglycol dodecyl ether, sarcosyl, sodium dodecyl sulfate, polyethoxysorbitan, deoxycholate, sodium octyl sulfate, sodium tetradecyl sulfate, sodium cholate, octylthioglucopyranoside, n-octylglucopyranoside, sodium bis (2-ethylhexyl) sulfosuccinate or combinations of two or more of the foregoing. A single surfactant maybe be used or a combination of two or more surfactants (e.g., at least two, at least 3, or 2 or 3 or 4 surfactants).

Where the desired protein contains disulfide bonds in the native conformation it is generally advantageous to include at least one disulfide shuffling agent pair in the mixture. The disulfide shuffling agent pair facilitates the breakage of strained non-native disulfide bonds and the reformation of native-disulfide bonds.

In general, the disulfide shuffling agent pair includes a reducing agent and an oxidizing agent. Exemplary oxidizing agents oxidized glutathione, cystine, cystamine, molecular oxygen, iodosobenzoic acid, sulfitolysis and peroxides. Exemplary reducing agents include glutathione, cysteine, cysteamine, diothiothreitol, dithioerythritol, tris(2-carboxyethyl)phosphine hydrochloride, or β-mercaptoethanol.

Exemplary disulfide shuffling agent pairs include oxidized/reduced glutathione, cystamine/cysteamine, and cysteine/cysteine.

Additional disulfide shuffling agent pairs are described by Gilbert H F. (1990). “Molecular and Cellular Aspects of Thiol Disulfide Exchange.” Advances in Enzymology and Related Areas of Molecular Biology 63:69-172, and Gilbert H F. (1995). “Thiol/Disulfide Exchange Equilibria and Disulfide Bond Stability.” Biothiols, Pt A. p 8-28, which are hereby incorporated by reference in their entirety.

The selection and concentration of the disulfide shuffling agent pair will depend upon the characteristics of the desired protein. Typically concentration of the disulfide shuffling agent pair taken together (including both oxidizing and reducing agent) is from about 0.1 mM to about 100 mM of the equivalent oxidized thiol, however, the concentration of the disulfide shuffling agent pair should be adjusted such that the presence of the pair is not the rate limiting step in disulfide bond rearrangement.

In some embodiments, the concentration will be about 1 mM, about 2 mM, about 3 mM about 5 mM, about 8 mM, about 9 mM, about 10 mM, about 20 mM, about 30 mM, about 40 mM, about 50 mM, about 60 mM, about 70 mM, about 80 mM, about 90 mM, about 100 mM, or from about 80 mM to about 100 mM, from about 0.1 mM to about 20 mM, from about 10 mM to about 50 mM, from about 1 mM to about 100 mM, from about 50 mM to about 100 mM, from about 20 mM to about 100 mM, from about 0.1 mM to about 10 mM, from about 0.1 mM to about 8 mM; from about 0.1 mM to about 6 mM, from about 0.1 mM to about 7 mM, from about 0.1 mM to about 5 mM, from about 0.1 mM to about 3 mM, from about 0.1 mM to about 1 mM.

A single disulfide shuffling agent pair maybe be used or a combination of two or more disulfide shuffling agent pairs (e.g., at least two, at least 3, or 2 or 3 or 4 disulfide shuffling agent pairs).

Chaotropic agents (also referred to as a “chaotrope”) are compounds, including, without limitation, guanidine, guanidine hydrochloride (guanidinium hydrochloride, GdmHCl), guanidine sulfate, urea, sodium thiocyanate, and/or other compounds which disrupt the noncovalent intermolecular bonding within the protein, permitting the polypeptide chain to assume a substantially random conformation

Chaotropic agents may be used in concentration of from about 10 mM to about 8 M. The optimal concentration of the chaotropic agent will depend on the desired protein as well as on the particular chaotropes selected. The choice of particular chaotropic agent and determination of optimal concentration can be optimized by the skilled artisan in view of the teachings provided herein.

In some embodiments, the concentration of the chaotropic agent will be, for example, from about 10 mM to about 8 M, from about 10 mM to about 7 M, from about 10 mM to about 6 M, from about 0.1 M to about 8 M, from about 0.1 M to about 7 M, from about 0.1 M to about 6 M, from about 0.1 M to about 5 M, from about 0.1 M to about 4 M, from about 0.1 M to about 3 M, from about 0.1 M to about 2 M, from about 0.1 M to about 1 M, from about 10 mM to about 4 M, from about 10 mM to about 3 M, from about 10 mM to about 2 M, from about 10 mM to about 1 M, or about, 10 mM, about 50 mM, about 75 mM, about 0.1 M, about 0.5 M, about 0.8 M, about 1 M, about 2 M, about 3 M, about 4 M, about 5 M, about 6 M, about 7 M, about 8 M.

When used in the present methods, it is often advantageous to use chaotropic agents in non-denaturing concentrations to facilitate the dissociation of hydrogen bonds. While a non-denaturing concentration will vary depending on the desired protein, the range of non-denaturing concentrations is typically from about 0.1 to about 4 M. In some embodiments the concentration is from about 0.1 M to about 2 M.

In certain embodiments, guanidine hydrochloride or urea are the chaotropic agents.

A single chaotropic agent maybe be used or a combination of two or more chaotropic agents (e.g., at least two, at least 3, or 2 or 3 or 4 chaotropic agents).

Agitation: Protein solutions can be agitated before and/or during refolding. Agitation can be performed by methods including, but not limited to, ultrasound energy (sonication), mechanical stirring, mechanical shaking, pumping through mixers, or via cascading solutions.

Temperature: The methods described herein can be performed at a range of temperature values, depending on the particular protein of interest. The optimal temperature, in concert with other factors, can be optimized as described herein. Proteins can be refolded at various temperatures, including at about room temperature, about 25° C., about 30° C., about 37° C., about 50° C., about 75° C., about 100° C., about 125° C., or ranges of from about 20 to about 125° C., about 25 to about 125° C., about 25 to about 100° C., about 25 to about 75 ° C., about 25 to about 50° C., about 50 to about 125° C., about 50 to about 100° C., about 50 to about 75 ° C., about 75 to about 125° C., about 5 to about 100° C., or about 100 to about 125° C.

In some embodiments of the methods, the temperature can range from about 0° C. to about 100° C. without adversely affecting the protein of interest. Thus in certain embodiments, the temperature may be from about 0° C. to about 75° C., from about 0° C. to about 55° C., from about 0° C. to about 35° C., from about 0° C. to about 25° C., from about 20° C. to about 75° C., from about 20° C. to about 65° C., from about 20° C. to about 35° C., from about 20° C. to about 25° C.

Although increased temperatures are often used to cause aggregation of proteins, when coupled with increased hydrostatic pressure it has been found that increased temperatures can enhance refolding recoveries effected by high pressure treatment, provided that the temperatures are not so high as to cause irreversible denaturation. Generally, the increased temperature for refolding should be about 20° C. lower than the temperatures at which irreversible loss of activity occurs. Relatively high temperatures (for example, about 60° C. to about 125° C., about 80° C. to about 110° C., including about 100° C., about 105° C., about 110° C., about 115° C., about 120° C. and about 125° C.) may be used while the solution is under pressure, as long as the temperature is reduced to a suitably low temperature before depressurizing. Such a suitably low temperature is defined as one below which thermally-induced denaturation or aggregation occurs at atmospheric conditions.

“High pressure” or “high hydrostatic pressure,” for the purposes of the invention is defined as pressures of from about 500 bar to about 10,000 bar.

In some embodiments, the increased hydrostatic pressure may be from about 500 bar to about 5000 bar, from about 500 bar to about 4000 bar, from about 500 bar to about 2000 bar, from about 500 bar to about 2500 bar, from about 500 bar to about 3000 bar, from about 500 bar to about 6000 bar, from about 1000 bar to about 5000 bar, from about 1000 bar to about 4000 bar, from about 1000 bar to about 2000 bar, from about 1000 bar to about 2500 bar, from about 1000 bar to about 3000 bar, from about 1000 bar to about 6000 bar, from about 1500 bar to about 5000 bar, from about 1500 bar to about 3000 bar, from about 1500 bar to about 4000 bar, from about 1500 bar to about 2000 bar, from about 2000 bar to about 5000 bar, from about 2000 bar to about 4000 bar, from about 2000 bar to about 3000 bar, or about 1000 bar, about 1500 bar, about 2000 bar, about 2500 bar, about 3000 bar, about 3500 bar, about 4000 bar, about 5000 bar, about 6000 bar, about 7000 bar, about 8000 bar, about 9000 bar.

Reduction of pressure: Where the reduction in pressure is performed in a continuous manner, the rate of pressure reduction can be constant or can be increased or decreased during the period in which the pressure is reduced. In some variations, the rate of pressure reduction is from about 5000 bar/1 sec to about 5000 bar/4 days (or about 3 days, about 2 days, about 1 day). Thus in some variations the rate of pressure reduction can be performed at a rate of from about 5000 bar/1 sec to about 5000 bar/80 hours, from about 5000 bar/1 sec to about 5000 bar/72 hours, from about 5000 bar/1 sec to about 5000 bar/60 hours, from about 5000 bar/1 sec to about 5000 bar/50 hours, from about 5000 bar/1 sec to about 5000 bar/48 hours, from about 5000 bar/1 sec to about 5000 bar/32 hours, from about 5000 bar/1 sec to about 5000 bar/24 hours, from about 5000 bar/1 sec to about 5000 bar/20 hours, from about 5000 bar/1 sec to about 5000 bar/18 hours, from about 5000 bar/1 sec to about 5000 bar/16 hours, from about 5000 bar/1 sec to about 5000 bar/12 hours, from about 5000 bar/1 sec to about 5000 bar/8 hours, from about 5000 bar/1 sec to about 5000 bar/4 hours, from about 5000 bar/1 sec to about 5000 bar/2 hours, from about 5000 bar/1 sec to about 5000 bar/1 hour, from about 5000 bar/1 sec to about 1000 bar/min, about 5000 bar/1 sec to about 500 bar/min, about 5000 bar/1 sec to about 300 bar/min, about 5000 bar/1 sec to about 250 bar/min, about 5000 bar/1 sec to about 200 bar/min, about 5000 bar/1 sec to about 150 bar/min, about 5000 bar/1 sec to about 100, about 5000 bar/1 sec to about 80 bar/min, about 5000 bar/1 sec to about 50 bar/min, or about 5000 bar/1 sec to about 10 bar/min. For example, about 10 bar/min, about 250 bar/5 minute, about 500 bar/5 minutes, about 1000 bar/5 minutes, about 250 bar/5 minutes, 2000 bar/50 hours, 3000 bar/50 hours, 40000 bar/50 hours, etc. In some embodiments, the pressure reduction may be approximately instantaneous, as in where pressure is released by simply opening the device in which the sample is contained and immediately releasing the pressure.

Where the reduction in pressure is performed in a stepwise manner, the process comprises dropping the pressure from the highest pressure used to at least a secondary level that is intermediate between the highest level and atmospheric pressure. The goal is to provide an incubation or hold period at or about this intermediate pressure zone that permits a protein to adopt a desired conformation.

In some embodiments, where there are at least two stepwise pressure reductions there may be a hold period at a constant pressure between intervening steps. The hold period may be from about 10 minutes to about 50 hours (or longer, depending on the nature of the protein of interest). In some embodiments, the hold period may be from about 2 to about 30 hours, from about 2 to about 24 hours, from about 2 to about 18 hours, from about 1 to about 10 hours, from about 1 to about 8 hours, from about 1 to about 6 hours, from about 2 to about 10 hours, from about 2 to about 8 hours, from about 2 to about 6 hours, or about 2 hours, about 6 hours, about 10 hours, about 20 hours, or about 30 hours, from about 2 to about 10 hours, from about 2 to about 8 hours, from about 2 to about 6 hours.

In some variations, the pressure reduction includes at least 2 stepwise reductions of pressure (e.g., highest pressure reduced to a second pressure reduced atmospheric pressure would be two stepwise reductions). In other embodiments the pressure reduction includes more than 2 stepwise pressure reductions (e.g., 3, 4, 5, 6, etc.). In some embodiments, there is at least 1 hold period. In certain embodiments there is more than one hold period (e.g., at least 2, at least 3, at least 4, at least 5 hold periods).

In some variations of the methods the constant pressure after an initial stepwise reduction may be at a hydrostatic pressure of from about 500 bar to about 5000 bar, from about 500 bar to about 4000 bar, from about 500 bar to about 2000 bar, from about 1000 bar to about 4000 bar, from about 1000 bar to about 3000 bar, from about 1000 bar to about 2000 bar, from about 1500 bar to about 4000 bar, from about 1500 bar to about 3000 bar, from about 2000 bar to about 4000 bar, or from about 2000 bar to about 3000 bar.

In particular variations, constant pressure after the stepwise reduction is from about four-fifths of the pressure immediately prior to the stepwise pressure reduction to about one-tenth of prior to the stepwise pressure reduction. For example, constant pressure is at a pressure of from about four-fifths to about one-fifth, from about two-thirds to about one-tenth, from about two-thirds to about one-fifth, from about two-thirds to about one-third, about one-half, or about one-quarter of the pressure immediately prior to the stepwise pressure reduction. Where there is more than one stepwise pressure reduction step, the pressure referred to is the pressure immediately before the last pressure reduction (e.g., where 2000 bar is reduced to 1000 bar is reduced to 500 bar, the pressure of 500 bar is one-half of the pressure immediately preceding the previous reduction (1000 bar)).

Where the pressure is reduced in a stepwise manner, the rate of pressure reduction (e.g., the period of pressure reduction prior to and after the hold period) may be in the same range as that rate of pressure reduction described for continuous reduction (e.g., in a non-stepwise manner). In essence, stepwise pressure reduction is the reduction of pressure in a continuous manner to an intermediate constant pressure, followed by a hold period and then a further reduction of pressure in a continuous manner. The periods of continuous pressure reduction prior to and after each hold period may be the same continuous rate for each period of continuous pressure reduction or each period may have a different reduction rate. In some variations, there are two periods of continuous pressure reduction and a hold period. In certain embodiments, each continuous pressure reduction period has the same rate of pressure reduction. In other embodiments, each period has a different rate of pressure reduction. In particular embodiments, the hold period is from about 8 to about 24 hours. In some embodiments, the hold period is from about 12 to about 18 hours. In particular embodiments, the hold period is about 16 hours.

Combinations of the above conditions: Various combinations and permutations of the condition above, such as agitation of the protein under high pressure at an elevated temperature in the presence of chaotropes and redox reagents, can be employed as desired for optimization of refolding yields.

### Optimization of Reaction Conditions

The optimum conditions for solubilization and refolding in the context of the methods described herein are a function of the characteristics of both the target polypeptide and the binding partner. In standard optimization experiments, the influence of pressure, pH, temperature, ionic strength, surfactants, chaotropes, stabilizing agents, and refolding time on refolding should be tested. Once the key process parameters are identified, a central composite design can be used to optimize the appropriate conditions for each parameter. Guidance regarding typical ranges for the various parameters is provided in more detail below.

Initial studies can be conducted to screen the effect of solution conditions, solution pH, redox environment, and high pressure treatment on the solubilization and/or refolding of proteins. Screening studies are typically conducted, but not limited to, empirical screens that examine step-wise the effect of processing conditions on yields. Synergistic effects between different parameters are not examined in these screening studies. Exemplary screening studies that can be conducted are as described for the cases of recombinant placental bikunin, recombinant growth hormone, and malaria pfs48 (see e.g., Seefeldt et al. Protein Science, v13 (10), 2639-2650 2004, St. John et al., Journal of Biological Chemistry, v276 (50), 46856-46864, 2001, Seefeldt, “High pressure refolding of protein aggregates: efficacy and thermodynamics,” Dept. of Chemical and Biological Engineering Thesis, (2004), the disclosures of which are herein incorporated by reference in their entirety, particularly with respect to the screening studies described therein). High pressure refolding studies of bikunin and growth hormone demonstrate the step-wise screening process for solution conditions (pH 5-9), temperature (0-60° C.), ionic strength (0-160 mM NaCl), non-denaturing concentrations of chaotropes (0-1.0 M urea or 0-2.0 M guanidine) and refolding time (0-24 hours). Studies can be conducted at about 2000 bar, about 2100 bar, about 2150 bar, etc. and compared to samples treated at atmospheric pressure. Other parameters, including those described herein, that can be screened include, but are not limited to, the presence and amount of stabilizing agents, surfactants, salts, etc., as described herein. It should be noted that statistical analysis of variance (ANOVA's) can be used to rapidly screen which solution parameters affect refolding yields. In addition to the teaching provided herein, U.S. 2004/0038333, Seefeldt et al. Protein Science, v13 (10), 2639-2650 2004, and St. John et al., Biotechnology Progress, v 18, (3), 565-571, 2002 (incorporated herein by reference in their entirety) also provide guidance regarding empirical screening procedures for determining the optimal solubilization and refolding conditions.

In this manner, the skilled artisan can determine the effect of processing conditions on the refolding of protein aggregates through the use of high pressure. It has been shown in the literature that refolding reactions can have interactions between the process conditions, which prevents single-variable screening from effectively optimizing the process. For instance, pH affects protein conformation stability, protein colloidal stability, and disulfide bond formation kinetics. To effectively optimize the effect of pH, or any other process parameter, studies need to be conducted to account for interactions. In these instances, statistical experimental designs need to be employed. As described herein, solubilization is also examined as a function of urea, by step-wise analysis in a range from 0-4.5M urea at pH 8.0. The effect of reduced and oxidized disulfide shuffling agents is screened step-wise as a function of reduced/oxidized ratio while arbitrarily setting the pH to 8.0, urea concentration to 1.5M, and protein concentration. Once the significant parameters are identified, a face-centered statistical designed experiment is used to optimize the refolding conditions, taking into account interactions.

After initial optimization studies are performed for the protein of interest, more granular optimization can be used to determined the optimal conditions for performing the solubilization and refolding processes. This process can generally be described as an experimental optimization that takes into account synergistic interactions between the critical parameters identified in the initial step-wise studies. An effective method for conducting these studies involves using a three or five level central composite statistical analysis, which takes into account interactions between the reaction parameters while minimizing the required number of experiments.

Another useful aid for optimizing conditions and/or monitoring solubilization or refolding is in situ spectroscopic measurement of samples under pressure. This is a well-known process for examining polypeptide stability under pressure, but it has been underutilized in protein aggregation studies. Using high pressure spectroscopic techniques to observe aggregate dissolution under pressure will help determine the optimal pressure ranges for recovering proteins from aggregates. Custom made high pressure cells have been routinely used for high pressure unfolding studies and can be adapted for use in high pressure disaggregation and refolding. Additional guidance for the skilled artisan may also be found in Paladini and Weber, Biochemistry, v 20 (9), 2587-2593, 1981 and Seefeldt et al. Protein Science, v 13 (10), 2639-2650 2004, incorporated by reference herein in their entirety.

Methods that can be employed to monitor the optimization of various parameters include Fourier Transform Infrared Spectroscopy (FTIR), circular dichroism (CD) spectroscopy (far and/or near UV), UV spectroscopy, measurement of total protein concentrations (e.g., BCA assay method (Pierce Chemical Co., Rockford, Ill.), etc), activity assays to measure the activity of the target polypeptide, electrophoretic gels with molecular weight markers to visualize the appearance of native protein under various conditions, HPLC analysis of soluble polypeptide fractions, etc.

Suitable devices for performing high pressure spectroscopy can be obtained commercially (e.g., such as fluorescence cells available from ISS Inc., Champaign, Ill. or fluorescence/ultraviolet absorbance cells available from BaroFold Inc., Boulder, Colo.) or can be fabricated by the skilled artisan. For example, Randolph et al., U.S. Patent Application Publication No. 2004/0038333, incorporated by reference herein in its entirety, described a high-pressure W spectroscopy cell made of stainless steel, sealed with Btma-N 90 durometer o-rings and with an optical port diameter of 6 mm and pathlength of 7.65 mm. The cell utilized cylindrical sapphire windows (16 mm diameter, 5.1 mm thick) and was capable of experiments up to 250 MPa. Separation of the sample from the pressure transmitting fluid was facilitated by a piston device external to the cell.

### High Pressure Devices and Considerations

Commercially available high pressure devices and reaction vessels, such as those described in the examples, may be used to achieve the hydrostatic pressures in accordance with the methods described herein (see BaroFold Inc., Boulder Colo.). Additionally devices, vessels and other materials for carrying out the methods described herein, as well as guidance regarding the performing increased pressure methods, are described in detail in U.S. Pat. No. 6,489,450, which is incorporated herein in its entirety. The skilled artisan is particularly directed to column 9, lines 39-62 and Examples 2-4. International Pat. App. Pub. No. WO 02/062827, incorporated herein in its entirety, also provides the skilled artisan with detailed teachings regarding devices and use thereof for high hydrostatic pressure solubilization of aggregates throughout the specification. Particular devices and teachings regarding the use of high pressure devices is also provided in U.S. Pat. App. Ser. No. 60/739,094 and International Patent Application No. PCT/US2006/045297, which are hereby incorporated by reference herein in their entirety.

Multiple-well sample holders may be used and can be conveniently sealed using self-adhesive plastic covers. The containers, or the entire multiple-well sample holder, may then be placed in a pressure vessel, such as those commercially available from the Flow International Corp. or High Pressure Equipment Co. The remainder of the interior volume of the high-pressure vessel may than be filled with water or other pressure transmitting fluid.

Mechanically, there are two primary methods of high-pressure processing: batch and continuous. Batch processes simply involve filling a specified chamber, pressurizing the chamber for a period of time, and repressurizing the batch. In contrast, continuous processes constantly feed aggregates into a pressure chamber and soluble, refolded proteins move out of the pressure chamber. In both set ups, good temperature and pressure control is essential, as fluctuations in these parameters can cause inconsistencies in yields. Both temperature and pressure should be measured inside the pressure chamber and properly controlled.

There are many methods for handling batch samples depending upon the specific stability issues of each target protein. Samples can be loaded directly into a pressure chamber, in which case the aqueous solution and/or suspension would be used as the pressure medium.

Alternately, samples can be loaded into any variety of sealed, flexible containers, including those described herein. This allows for greater flexibility in the pressure medium, as well as the surfaces to which the mixture is exposed. Sample vessels could conceivably even act to protect the desired protein from chemical degradation (e.g., oxygen scavenging plastics are available).

With continuous processing, scale-up is simple. Small volumes under pressure can be used to refold large volumes the sample mixture. In addition, using an appropriate filter on the outlet of a continuous process will selectively release soluble desired protein from the chamber while retaining both soluble and insoluble aggregates.

Pressurization is a process of increasing the pressure (usually from atmospheric or ambient pressure) to a higher pressure. Pressurization takes place over a predetermined period of time, ranging from 0.1 second to 10 hours. Such times include 1 second, 2 seconds, 5 seconds, 10 seconds, 20 seconds, 1 minute, 2 minutes, 5 minutes, to minutes, 30 minutes, 60 minutes, 2 hours, 3 hours, 4 hours, and 5 hours.

Repressurization is a process of decreasing the pressure, from a high pressure, to a lower pressure (usually atmospheric or ambient pressure). Depressurization takes place over a predetermined period of time, ranging from 10 seconds to 10 hours, and may be interrupted at one or more points to permit optimal refolding at intermediate (but still increased 30 compared to ambient) pressure levels. The repressurization or interruptions may be 1 second, 2 seconds, 5 seconds, 10 seconds, 20 seconds, 1 minute, 2 minutes, 5 minutes, 10 minutes, 30 minutes, 60 minutes, 2 hours, 3 hours, 4 hours, and 5 hours.

Degassing is the removal of gases dissolved in solutions and is often advantageous in the practice of the methods described herein. Gas is much more soluble in liquids at high pressure as compared to atmospheric pressure and, consequently, any gas headspace in a sample will be driven into solution upon pressurization. The consequences are two-fold: the additional oxygen in solution may chemically degrade the protein product, and gas exiting solution upon repressurization may cause additional aggregation. Thus, samples should be prepared with degassed solutions and all headspace should be filled with liquid prior to pressurization.

### EXAMPLES

### Example 1

### Optimization of Folding Conditions

Examples of screening studies that can be conducted are described for the cases of recombinant placental bikunin, recombinant growth hormone, and malaria pfs48. (Seefeldt et al. Protein Science, v 13 (10), 2639-2650 2004; St. John et al., Journal of Biological Chemistry, v 276 (50), 46856-46864, 2001; Seefeldt, Matthew B., “High pressure refolding of protein aggregates: efficacy and thermodynamics,” Ph.D. dissertation, University of Colorado at Boulder, 2004)). High pressure refolding studies of bikunin and growth hormone demonstrate the step-wise screening of solution conditions (pH 5-9), temperature (0-60 C), ionic strength (0-160 mM) NaCl, non-denaturing concentrations of chaotropes ((0-1M urea or 0-2M guanidine) and refolding time (0-24 hours). Other parameters that could be screened include, but are not limited to, the presence and amount of stabilizing agents, surfactants, salts, and other parameters as described herein. It should be noted that statistical analysis of variance (ANOVA's) can be used to rapidly screen the solution parameters that affect protein refolding. In addition to the teaching provided herein, U.S. 2004/0038333, Seefeldt et al. Protein Science, v 13 (10), 2639-2650 2004, St. John et al., Biotechnology Progress, v 18, (3), 565-571, 2002 (incorporated herein by reference in their entirety) also provide guidance regarding empirical screening procedures for determining the optimal solubilization and refolding conditions.

In this manner, the skilled artisan can determine the effect of process conditions on the refolding of protein aggregates through the use of high pressure. Alterations in one process condition can perturb the effect of other process conditions, which prevents optimization of the process through single-variable screening. For example, pH affects protein conformation stability, protein colloidal stability, and disulfide bond formation kinetics. To effectively optimize the effect of pH, or any other process parameter, studies need to be conducted to account for interactions of the parameter varied on other process parameters. In these instances, statistical experimental designs can be employed.

### Example 2

### Co-Refolding of Recombinant Protein of Interest (Protein X) in the Presence of a Binding Protein (Protein Y)

High pressure-solubilized Protein X is added to inclusion bodies of Protein Y to determine the feasibility of co-refolding these two proteins simultaneously, in order to test the hypothesis that the presence of the partner protein favors the formation of native protein structures and improves refolding yields. The co-refold solution conditions are identified by combining the optimized refolding conditions for Protein X and Protein Y as determined individually.

Inclusion bodies of Protein X are solubilized (and, if expressed as a fusion protein, cleaved) at pressure at or near 2000 bar as a function of refolding solution containing buffers, stabilizing agents, and redox agents. The pressure, temperature, depressurization rate, protein concentration, and time of the reaction are optimized individually for each protein. At this step, Protein X contains native structure, denatured structure, or may be disaggregated but in a non-native structure due to the presence of non-native disulfide bonds or the absence of disulfide bonds if the solution is reduced. Protein X is then added to the inclusion bodies of Protein Y in a refolding solution containing buffers, stabilizing agents, and redox agents. The pressure, temperature, depressurization rate, protein concentration, and time of the reaction is optimized for the co-refolding reaction. The conditions lead to the co-refolding of the two molecules. It should be noted that neither Protein X or Protein Y is present in its native, refolded state at the start of the experiment.

Protein X and Protein Y refolding solutions are prepared by the dilution of stock solutions into microcentrifuge tubes with 500 uL final volumes. All sample preparation is preferably conducted within a nitrogen hood to reduce oxygen partial pressures to no greater than 1% of atmospheric levels. To ensure sample integrity, urea and redox stock solutions are made fresh prior to use. All other stock solutions are stored at 4° C. For preparing samples, reagents are added in the following order: Buffer, chaotrope, stabilizing agents, other non-oxidizing compounds, oxidized disulfide shuffling agent, reduced disulfide shuffling agent, water. The sample is then mixed and the appropriate amount of protein was added, followed by further mixing. The samples are loaded into sealed syringe high pressure refolding devices for pressure treatment and atmospheric controls. The sealed syringe high pressure refolding devices, described as variable volume sample vials, (one per sample) are prepared by heat sealing the Luer end of a 1 ml polypropylene syringe (LUER-LOK is a registered trademark of Becton, Dickinson and Co., Franklin Lakes, N.J. for interlocking tubing and syringe seals), and numbered for identification. The end of the syringe is trimmed so that the final volume of the syringe is 800 uL. Samples are prepared in 1.5 ml microcentrifuge tubes as previously described and then placed in the heat-sealed syringes. The syringes are then sealed with the plunger, using a 21 gauge needle along the side of the plunger to vent all air from the sample. At that point, samples are ready for pressure treatment. The samples are removed from the nitrogen hood and vented to remove all residual nitrogen gas present in the samples. Co-refold studies are conducted by first solubilizing Protein X using pressure treatment as a function of protein concentration. This material is then diluted into refolding buffer containing Protein Y inclusion bodies and pressure treated to begin refolding. A two-step process can be employed if the presence of certain agents in the refolding buffer for Protein X inhibits the refolding of Protein Y, or if the pressures for optimal refolding of the proteins differ. Manually driven, 4000 bar high pressure generators are used with water as a pressure transmitting fluid. The sealed syringes are placed in custom-built pressure cells and pressurized to the appropriate pressure over a period of two to five minutes. The refolding reaction can be conducted overnight (typically sixteen hours). For depressurization, the pressure is reduced by 250 bar every five minutes until atmospheric pressure was reached. All testing is conducted at the appropriate temperature. The samples are then removed from the pressure vessels and syringes and placed into microcentrifuge tubes in the nitrogen hood to prevent oxidation. Samples are then analyzed by RP-HPLC within three hours after depressurization for sample analysis/chromatography, peak integration, and yield analysis. The samples are kept at 4° C. at all times.

Sample analysis/chromatography, peak integration, and yield analysis can be conducted by reverse phase HPLC, e.g., on an analytical column, using gradient elution with standard solvents such as 0.1% TFA (solvent A) in water and 0.1% TFA (solvent B) in acetonitrile with protein detection at 220 nm. Packing material such as POROS® R2/10 can be used (POROS is a registered trademark of Perseptive Biosystems, Inc., Framingham Mass., for a chromatography medium.) The percentage of the recombinant protein of interest that is refolded is calculated by standard methods, such as peak integration and comparison.

### Example 3

### Pressure-Modulated Refolding of Inclusion Bodies which Bind to a Polymeric Ligand

Two alternate methods for sample preparation can be used, depending on the disulfide bond configuration of the starting inclusion bodies. If the inclusion bodies are monomerically sized as analyzed by non-reduced SDS-PAGE, samples can be prepared as described in the preceding example. If the starting inclusion bodies are crosslinked with non-native intermolecular disulfide bonds, refolding is conducted with dialysis, as described below, or solution exchange under pressure, as described in International Patent Application No. PCT/US2006/045297.

Dialysis under pressure is conducted by taking approximately 6 inches of dialysis tubing per sample, prepared by placing SpectraPor regenerated cellulose dialysis membrane, 3.5 KDa cutoff, 8 mm flat width (Spectrum Labs product #133108) into a beaker with about 250 ml purified water (Sigma) for at least one hour. If frozen, inclusion bodies of the protein to be refolded are thawed. Dialysis chambers (one per sample) are prepared by heat sealing the Luer end of a syringe (LUER-LOK is a registered trademark of Becton, Dickinson and Co., Franklin Lakes, N.J. for interlocking tubing and syringe seals), and numbered for identification. Dialysis buffers are prepared in 50 mL screwcap tubes, omitting addition of oxidizing redox agents, but containing reducing agents to facilitate the reduction of non-native disulfide bonds present in the starting inclusion body material. Samples are prepared with the omission of reducing agent and protein. Oxidizing agents are then added to the dialysis buffers immediately prior to starting the experiment to prevent oxidation during sample preparation. The pH is checked using a calibrated pH meter. If the pH is incorrect, it is adjusted to the desired pH with a minimal volume of concentrated NaOH or HCl. Reducing agents (e.g., DTT) are then added to the sample tubes, followed by mixing to minimize undesired oxidation. The inclusion body suspension is then vortexed, followed by transfer of the appropriate volume to each tube, which is then again vortexed to mix. 600 uL of each well-mixed sample is quickly transferred into a knotted piece of dialysis tubing. Any air bubbles present are squeezed out of the tubing before carefully knotting the open end of the dialysis membrane and placing the tubing into the appropriately-numbered syringe. After all samples are transferred to dialysis tubing and placed in their syringes, the appropriate dialysis buffer is added to fill the syringe. The syringes are then sealed with the plunger, using a needle to vent any air bubbles. At that point, samples are ready for pressure treatment.

Refolding conditions are determined by the methods described previously in example 1. The refolding buffer, containing the protein to be refolded and the polymeric ligand (e.g., a polyanion such as dextran sulfate) is refolded in a solution condition containing, but not limited to, stabilizing agents, surfactants, salts, or other components, as described herein.

Studies are conducted at both atmospheric and high hydrostatic pressure to confirm that a higher yield is observed during refolding at high hydrostatic pressure. Studies are conducted as a function of both the concentration of the protein to be refolded and the polymeric ligand concentration, using methods described previously.

The disclosures of all publications, patents, patent applications and published patent applications referred to herein by an identifying citation are hereby incorporated herein by reference in their entirety. U.S. Pat. No. 6,489,450 and Pat. No. 7,064,192, and United States Patent Application Publication Nos. 2004/0038333 and 2006/0188970 are specifically incorporated herein by reference in their entirety. In particular, the exemplary proteins for refolding and the experimental techniques for refolding found in those documents are incorporated by reference herein.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

What is claimed is:

1. A method for producing biologically active protein from a mixture comprising aggregated or denatured protein, comprising:
a) adding a specific binding agent for the biologically active protein to the mixture;
b) subjecting the mixture to high hydrostatic pressure for a period of time sufficient to form biologically active protein; and
c) reducing the pressure to atmospheric pressure.

2. The method of claim 1, wherein the specific binding agent is a small organic molecule.

3. The method of claim 2, wherein the small organic molecule is a rigid molecule.

4. The method of claim 2, wherein the small organic molecule is a flexible molecule.

5. The method of claim 1, wherein the specific binding agent is a polypeptide.

6. The method of claim 1, wherein the specific binding agent is a nucleic acid molecule.

7. A method for producing biologically active protein from a mixture comprising aggregated or denatured protein, comprising:
a) adding a homopolymer or non-naturally-occurring polymer which binds to the biologically active protein to the mixture;
b) subjecting the mixture to high hydrostatic pressure for a period of time sufficient to form biologically active protein; and
c) reducing the pressure to atmospheric pressure.

8. The method of claim 7, wherein the homopolymer or non-naturally occurring polymer binds specifically to the biologically active protein.

9. The method of claim 7, wherein the homopolymer or non-naturally occurring polymer binds preferentially to the biologically active protein over inactive, denatured, or aggregated protein.

10. The method of claim 9, wherein the homopolymer or non-naturally occurring polymer binds preferentially to the biologically active protein via electrostatic interaction.

11. The method of claim 9, wherein the homopolymer or non-naturally occurring polymer binds preferentially to the biologically active protein via hydrophobic interaction.

12. The method of claim 9, wherein the homopolymer or non-naturally occurring polymer is a homopolymer.

13. The method of claim 9, wherein the homopolymer or non-naturally occurring polymer is a non-naturally occurring polymer.

14. The method of claim 9, wherein the homopolymer or non-naturally occurring polymer is dextran sulfate.

15. A method for producing biologically active protein from a mixture comprising a first aggregated or denatured protein, comprising:
a) adding a second aggregated or denatured protein to the mixture; and
b) subjecting the mixture to high hydrostatic pressure for a period of time sufficient to form biologically active protein, wherein said first and second aggregated or denatured proteins specifically interact under high pressure; and
c) reducing the pressure to atmospheric pressure.

16. The method of claim 15, further comprising:
d) separating the first and second proteins.

17. The method of claim 16, wherein the separating of the first and second proteins is performed by affinity chromatography, HPLC, dialysis, ion exchange chromatography, size exclusion chromatography, reverse-phase chromatography, ammonium sulfate precipitation, or electrophoresis.

18. The method of claim 15, wherein one of the first or second proteins is a chaperone protein.

19. The method of claim 15, wherein the first and second proteins continue to interact after the pressure is reduced to atmospheric pressure.

20. The method of claim 19, wherein the first and second proteins form a heterodimer in their biologically active state.

21. The method of claim 19, wherein one of the first and second proteins is an enzyme and the other is a substrate for the enzyme.

22. The method of claim 19, wherein one of the first and second proteins is an enzyme and the other is an inhibitor of the enzyme.

23. The method of claim 19, wherein one of the first and second proteins is an enzyme and the other is a regulator or modulator of the enzyme.

24. The method of claim 19, wherein one of the first and second proteins is a receptor and the other is a ligand for the receptor.

25. The method of claim 24, wherein one of the first and second proteins is a receptor and the other is an agonist for the receptor.

26. The method of claim 24, wherein one of the first and second proteins is a receptor and the other is an antagonist for the receptor.
